(19) United States
(12) Patent Application Publication
(10) Pub. No.: US 2019/0302111 A1
(43) Pub. Date: Oct. 3, 2019

(54) DETECTION KIT AND DETECTION METHOD

(71) Applicant: CANON KABUSHIKI KAISHA, Tokyo (JP)

(72) Inventors: Tatsuo Takeuchi, Yokohama-shi (JP); Masaru Sugita, Tokyo (JP); Keigo Mizusawa, Kawasaki-shi (JP); Ryuji Higashi, Kawasaki-shi (JP); Koichi Suzuki, Yokohama-shi (JP)

(21) Appl. No.: 16/360,398

(22) PCT Filed: Sep. 26, 2017
(86) PCT No.: PCT/JP2017/034764

§ 371 Date: Mar. 21, 2019

(30) Foreign Application Priority Data
Sep. 30, 2016 (JP) 2016-193554

(51) Int. Cl. G01N 33/558; G01N 33/74

(52) U.S. Cl. CPC G01N 33/558; G01N 33/74

(57) ABSTRACT

A detection kit for a detection target, including, on a support, an introduction portion into which a sample containing the detection target is introduced, a plurality of mixing portions each configured to provide a mixture of the sample that is introduced into the introduction portion and that is distributed and a signaling substance that can provide a signal, and bonding portions each including a bonding substance that can be selectively bonded to the detection target and the signaling substance in each of the mixtures provided in the plurality of mixing portions, wherein the amounts or concentrations of the signaling substance disposed in the plurality of mixing portions are different from each other.

### CROSS-REFERENCE TO RELATED APPLICATIONS

This application is a continuation of International Patent Application No. PCT/JP2017/034764, filed Sep. 26, 2017, which claims the benefit of Japanese Patent Application No. 2016-193554, filed Sep. 30, 2016, both of which are hereby incorporated by reference herein in their entirety.

### TECHNICAL FIELD

The present invention provides a detection kit for a detection target and a detection method for detecting a detection target.

### BACKGROUND ART

To date, methods and apparatuses for obtaining vital signs of living bodies by measuring the presence or absence and amounts of biomarkers, which are living body metabolites contained in blood, urine, tears, or the like, have been developed. In general, to obtain such vital signs, various test samples taken from living bodies have been subjected to chemical analysis, and compositions and amounts thereof have been measured, by using techniques such as liquid chromatography. However, such apparatuses are very large, and simple detection methods and apparatuses have been desired.

Japanese Patent Laid-Open No. 2008-32494 (PTL 1) discloses a measuring kit that simply measures a target substance contained in a test sample. Regarding the detection kit disclosed in PTL 1, a mixture in which a test sample containing a target substance is mixed with a competitive substance is sequentially brought into contact with a competitive capture substance fixed to the kit and a determination substance fixed to the kit. Subsequently, the amount of target substance is measured by measuring the amount of reaction product obtained from the competitive substance bonded to and captured by the determination substance. That is, when the competitive substance is assumed to be an antibody that can be bonded to the target substance and the competitive capture substance is assumed to be the target substance or an analogue thereto, in the case in which the amount of target substance is sparse, the competitive substance is bonded to and captured by the fixed competitive capture substance and the amount of the competitive substance that reaches the determination substance decreases. As a result, the amount of reaction product obtained from the competitive substance bonded to and captured by the determination substance decreases. On the other hand, as the amount of the target substance increases, the amount of the competitive substance that is bonded to the target substance and that is not bonded to the fixed competitive capture substance increases and, therefore, the amount of the competitive substance that reaches the determination substance increases. As a result, the amount of reaction product obtained from the competitive substance bonded to and captured by the determination substance increases. Consequently, qualitative information with respect to the amount of the target substance can be obtained.

However, it is necessary that not only the competitive capture substance but also the determination substance be fixed to the detection kit disclosed in PTL 1 and, therefore, the configuration is complex. In addition, the competitive substance is limited to a substance that can be bonded to both the target substance and the determination substance and, therefore, restrictions placed on the degree of freedom in kit design increase. Therefore, a detection target detection kit having a simpler configuration and higher degree of freedom in design than before has been desired.

### CITATION LIST

### Patent Literature

PTL 1 Japanese Patent Laid-Open No. 2008-32494

The present invention was realized in consideration of the above-described examples in the related art, and it is an object to provide a detection target detection kit having a simpler configuration and higher degree of freedom in design than before.

### SUMMARY OF INVENTION

A detection kit according to the present invention is a detection kit for a detection target and includes, on a support, an introduction portion into which a sample containing the detection target is introduced, a plurality of mixing portions each configured to provide a mixture of the sample that is introduced into the introduction portion and that is distributed and a signaling substance that can provide a signal, and bonding portions each including a bonding substance that can be selectively bonded to the detection target and the signaling substance in each of the mixtures provided in the plurality of mixing portions, wherein the amounts or concentrations of the signaling substance disposed in the plurality of mixing portions are different from each other.

Further features of the present invention will become apparent from the following description of exemplary embodiments with reference to the attached drawings.

### BRIEF DESCRIPTION OF DRAWINGS

FIG. 1 is a top view illustrating a detection kit according to an embodiment and example 1 of the present invention.

FIG. 2 is a sectional view illustrating the detection kit according to the embodiment and example 1 of the present invention (cross section of the top view shown in FIG. 1).

FIG. 3 is a sectional view illustrating a detection kit according to example 2 of the present invention.

FIG. 4 is a diagram showing an example of a molecular structure of a detection target in examples 1 and 2 of the present invention.

FIG. 5 is a diagram showing an example of a molecular structure of a signaling substance in examples 1 and 2 of the present invention.

### DESCRIPTION OF EMBODIMENTS

Detection Kit

A detection kit according to an embodiment of the present invention will be described, but the present invention is not limited to that described below.

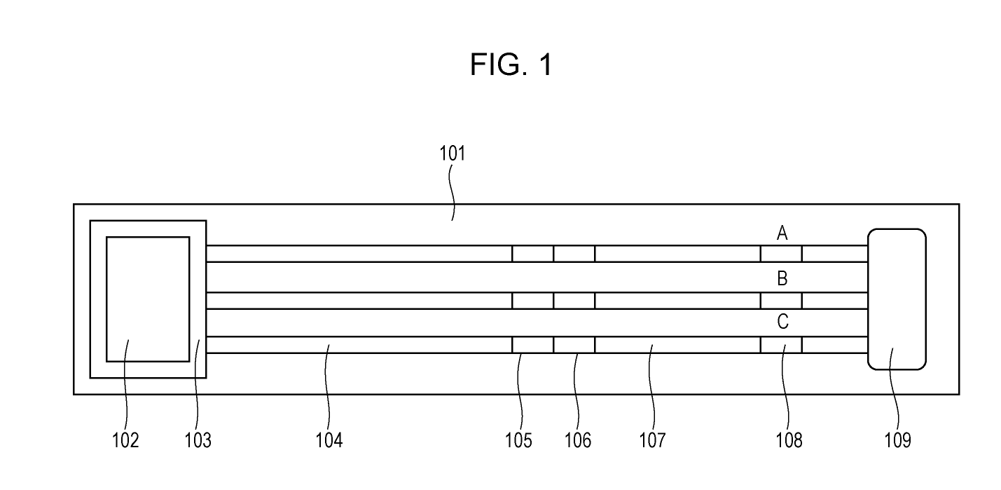

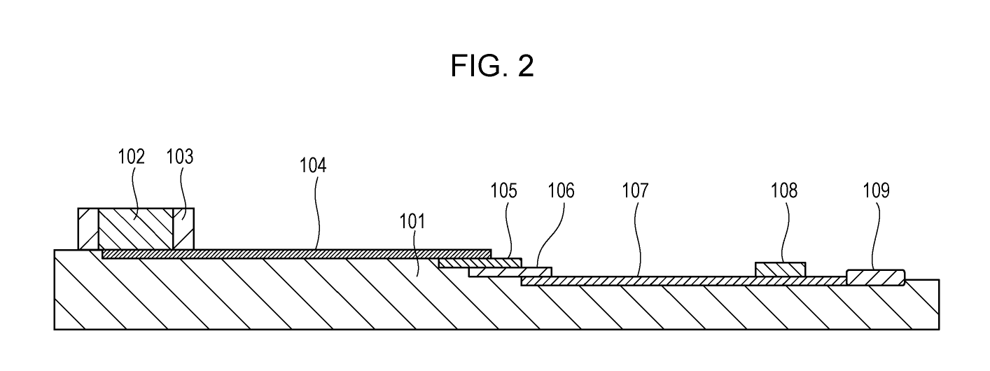

An example of the detection kit according to the present embodiment will be described with reference to FIGS. 1 and 2. FIG. 1 is a top view of the detection kit according to the present embodiment, and FIG. 2 is a sectional view of the detection kit shown in FIG. 1.

The detection kit according to the present embodiment includes at least an introduction portion 102, a mixing portion 105, and a bonding portion 108 on a support 101. A sample containing a detection target that is a target of semi-quantification is introduced into the introduction portion 102. A plurality of mixing portions 105 are disposed on the support 101 for the purpose of providing a mixture of each distributed sample and a signaling substance that can provide a signal. A bonding substance that can be selectively bonded to the detection target and the signaling substance is disposed in each of a plurality of bonding portions 108. Each of the plurality of bonding portions 108 is configured to include a fixed bonding substance such that the mixture provided in each of the plurality of mixing portions 105 comes into contact with the bonding substance.

As described above, the detection kit according to the present embodiment has a simple configuration in which at least the bonding substance has to be fixed to the support. In this regard, the bonding substance has only to be bondable to the detection target and the signaling substance, and the degree of freedom in design is high.

The detection kit according to the present embodiment will be described in more detail. The introduction portion 102 is located upstream, a checking portion 109 is located downstream, and the sample moves in the upstream to downstream direction.

The sample introduced into the introduction portion 102 is absorbed by an absorption portion 103 and distributed to a plurality of first transportation portions 104. It is preferable that the sample introduced into the introduction portion 102 be distributed to the plurality of first transportation portions 104 in equal amounts. However, even when the amounts of the samples distributed to the plurality of first transportation portions 104 are not equal to each other, it is sufficient that the amounts of the samples reaching the plurality of mixing portions 105 be equal to each other. The absorption portion 103 plays a role in not only facilitating the amounts of the sample distributed to the plurality of first transportation portions 104 becoming equal to each other but also absorbing any sample overflowing the introduction portion so as to suppress diffusion. In this regard, “the amounts of the sample become equal to each other” includes not only the case of perfectly the same but also the case in which the value of the amount of one sample falls within the range of 0.95-times to 1.05-times the value of the amount of the other sample. In a preferable case, the value of the amount of one sample falls within the range of 0.99-times to 1.01-times the value of the amount of the other sample.

Each sample that passes through each of the plurality of first transportation portions 104 reaches each of the plurality of mixing portions 105. The sample is mixed with the signaling substance disposed in each of the plurality of mixing portions 105 so as to provide a mixture.

In this regard, the amounts or concentrations of the signaling substance disposed in the plurality of mixing portions 105 are different from each other. The case in which the amounts are different from each other will be described below. However, adjustment of the concentrations is convenient in, for example, the case in which the signaling substance is disposed in the form of being contained in a solvent.

The ratios of the detection target to the signaling substance in the mixtures provided in the plurality of mixing portions 105 are different from each other because the amounts of the signaling substance are different from each other. For example, the amount of the signaling substance disposed in the mixing portion 105 connected to A of the bonding portion 108 described later is set to be the largest, the amount of the signaling substance disposed in the mixing portion 105 connected to B is set to be the next largest, and the amount of the signaling substance disposed in the mixing portion 105 connected to C is set to be the smallest.

The detection target and the signaling substance in the mixture are dispersed and mixed with each other during movement from the mixing portion 105 through a development portion 106 to a second transportation portion 107. The mixtures that pass through the plurality of development portions 106 reach the bonding portions 108 through the plurality of second transportation portions 107. The detection target and the signaling substance in the mixture are selectively bonded to the bonding substance disposed in each of the plurality of bonding portions 108 (A, B, and C). The ratios of the detection target to the signaling substance in the mixtures that reach A, B, and C of the plurality of bonding portions 108 are different from each other. In the present example, of the samples that reach the bonding portions 108, the decreasing order of the ratio of the signaling substance to the detection target is A, B, and C. Consequently, regarding the magnitude of signals provided by the signaling substance in the bonding portions 108, the decreasing order of the magnitude of the signals is A, B, and C. In the case in which the amount of the detection target is small, a large amount of signaling substance is bonded to the bonding substance disposed in the bonding portion 108 and, therefore, a large signal is provided. Meanwhile, in the case in which the degree of bondability to the bonding substance (coupling constant) of the detection target is equal to that of the signaling substance, when the detection target increases, the amount of the detection target bonded to the bonding substance increases, and as a result, a provided signal becomes small. In this regard, “the coupling constants are equal” includes not only the case of perfectly the same but also the case in which the value of the coupling constant of one substance falls within the range of 0.95-times to 1.05-times the value of the coupling constant of the other substance. In a preferable case, the value of the coupling constant of one substance falls within the range of 0.99-times to 1.01-times the value of the coupling constant of the other substance.

In this manner, qualitative information of the amount of the detection target contained in the sample introduced into the introduction portion 102 is obtained. In this regard, “the coupling constants are equal” may denote such a degree that the effect of the present invention is obtained, that is, that the detection target and the signaling substance are competitively bonded to the bonding substance.

Next, the reason the detection kit according to the present invention can semi-quantitatively detect the detection target in the sample will be described with reference to an example. Easy-to-understand numerical values are adopted as various numerical values described here for the sake of facilitating understanding of the present embodiment. In addition, the magnitude of a signal is an example for describing the qualitative relationship and, therefore, no units are given.

The numbers of signaling substances disposed in the mixing portions 105 connected to A, B, and C of the bonding portions 108 are assumed to be 1000, 100, and 10, respectively. It is assumed that 10 bonding substances are disposed in each of A, B, and C of the bonding portions 108. In addition, the degree of bondability to the bonding substance (coupling constant) of the detection target is assumed to be equal to that of the signaling substance.

Initially, when the number of the detection targets is 10, it is conjectured that about 10, about 10, and about 5 signaling substances are bonded to the bonding substances of A, B, and C, respectively, of the bonding portions 108. Therefore, the ratio of the magnitude of the signal of A:B:C becomes roughly 100:10:5.

Next, when the number of the detection targets is 100, it is conjectured that about 10, 5, and 0 to 1 signaling substances are bonded to the bonding substances of A, B, and C, respectively, of the bonding portions 108. Therefore, the ratio of the magnitude of the signal of A:B:C becomes roughly 10:5:(0 to 1).

Finally, when the number of the detection targets is 1000, it is conjectured that 5, 0 to 1, and 0 to 1 signaling substances are bonded to the bonding substances of A, B, and C, respectively, of the bonding portions 108. Therefore, the ratio of the magnitude of the signal of A:B:C becomes roughly 5:(0 to 1):(0 to 1).

Therefore, in the case in which the amount of the detection targets is small, a signal is provided from each of A, B, and C. When the amount of the detection targets increases, almost no signal is provided from C of the bonding portions 108, and then, no signal is provided from B of the bonding portions 108. When the amount of the detection targets increases significantly, most of the substances that are bonded to the bonding substances are the detection targets, and no signal is provided from A of the bonding portions 108. In the case in which a large amount of detection targets is a sign of disease of a living body or indicates that there is high possibility of great stress, when a larger signal is provided from the detection kit according to the present embodiment, a high possibility of morbidity or great stress is indicated.

Further, when the amounts or concentrations of signaling substance disposed in A, B, and C of the mixing portions 105 differ from each other by, for example, an exponent of 10 (differ by an order of magnitude), a change in the amount of the detection target by about an order of magnitude can be detected on the basis of a change in the magnitude of signals provided from A, B, and C. That is, it can be said that the detection kit according to the present embodiment is a semi-quantitative system provided with some degree of quantitativeness that is not limited to qualitative information such as whether the amount of the detection target is large or small. In this regard, the range of the measurable amount of the detection target can be increased by differentiating the amounts or concentrations of signaling substance disposed in A, B, and C of the mixing portions 105 from each other by at least an order of magnitude. The remainder of the mixture (sample) that passes through the bonding portions 108 reaches the checking portion 109. The checking portion 109 checks that the sample has passed each section in the detection kit and, in addition, can prevent any remaining sample from leaking.

The detection kit according to the present embodiment includes three each of the first transportation portion, the mixing portion, the development portion, the second transportation portion, and the bonding portion, but the number is not limited to three. A form of two or a form of four or more may be adopted.

Next, specific examples of each section constituting the detection kit according to the present embodiment, a material for each member constituting the section, and the like will be described.

Detection Target

Examples of the detection target according to the present embodiment include a biomarker related to disease of a living body, physical condition, a degree of stress applied to a living body, or the like. For example, the target is a low-molecular-weight substance having a molecular weight of 1,000 or less among hormone-based substances metabolized in a living body. Specific examples include steroid-based hormones, amine-based hormones, nucleosides, and analogues thereof. In addition, antigens and antibodies may be adopted. In this case, any one of the detection target and the bonding substance is an antigen and the other is an antibody.

Further, of artificially synthesized low-molecular-weight substances, substances metabolized in a living body may also be adopted. In addition, substances that cause serious disease when taken from outside a living body such as low-molecular-weight toxins, for example, shellfish toxin represented by tetrodotoxin, may also be adopted.

It is preferable that the detection target be contained in blood, sweat, tears, or the like.

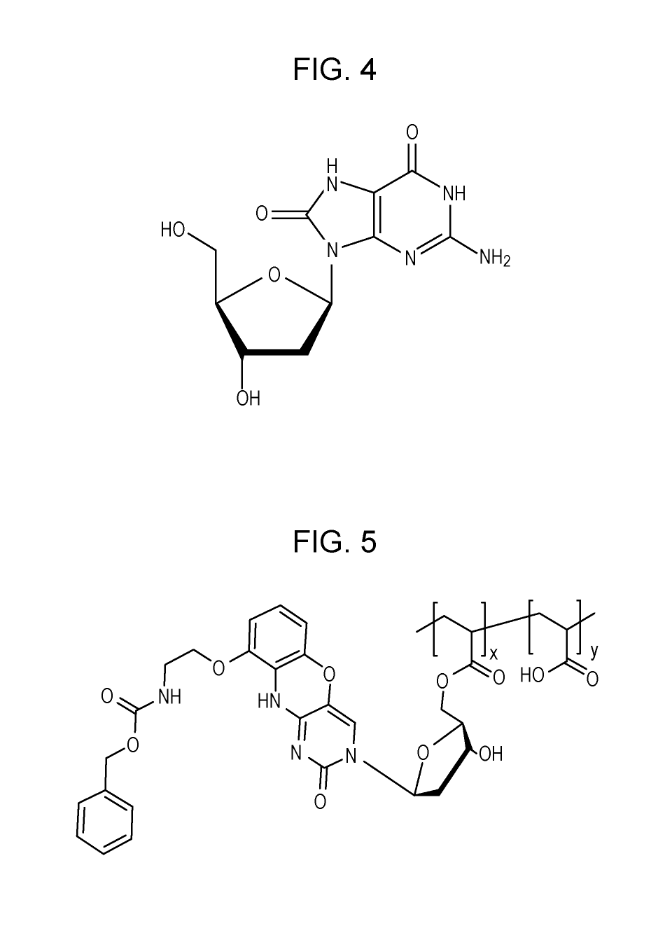

Examples of the detection target according to the present embodiment include 8-oxo-2′-deoxyguanosine (hereafter may be abbreviated as 8OHdG) having a structure shown in FIG. 4.

Sample

Examples of the sample containing the detection target include urine, blood, sweat, tears, and a liquid sample based on these. Such samples contain biomarkers, and vital signs of the living bodies are obtained by measuring the amounts of the biomarkers.

Meanwhile, regarding low-molecular-weight toxins, examples of samples include mucous membranes, mucus, and seawater that are considered to contain low-molecular-weight toxins and diluted solutions or concentrated solutions of these in which the amount of water contained is increased or decreased.

Support

Plastic, glass, film, or the like is used as a material for forming the support 101 and appropriately selected in accordance with the type of the sample, the type of the detection target, and the like. Regarding the dimension and the shape of the support 101, a square with a length in the movement direction of the sample of 3 cm to 5 cm and a width of 2 cm to 3 cm is preferable from the viewpoint of ease of handling, but the width may be increased in accordance with the type of the detection target, the amount and the concentration of the signaling substance, and the number of the first transportation portions 104. That is, the support has preferably a palm-size stick shape from the viewpoint of ease of use.

Introduction Portion

Examples of the material for the member constituting the introduction portion 102 of the sample include cellulose filter paper, glass fiber, polyurethane, polyacetate, cellulose acetate, nylon, and cotton cloth which have uniform characteristics. In particular, open-cell polyurethane foam can be appropriately selected in accordance with the solvent of the sample because the hydrophilicity or the hydrophobicity is readily adjusted. In the case of an aqueous sample, if a highly hydrophilic introduction portion is used, adsorption occurs and it is possible that the sample does not smoothly move to the downstream bonding portion 108. It is preferable that the material for forming the introduction portion be selected after distinguishing whether the sample is hydrophilic or hydrophobic such that a hydrophobic introduction portion is selected when the sample is hydrophilic or a hydrophilic introduction portion is selected when the sample is hydrophobic. Meanwhile, in the case in which the introduction portion 102 has a structure contained in the detection target and a hydrogen-bondable site, it is particularly preferable that treatment to suppress non-specific adsorption be performed. Examples of the treatment to suppress non-specific adsorption include the case in which a surfactant is disposed and the case in which inactive protein is disposed, and the treatment method may be selected in accordance with the characteristics of the detection target in the sample.

Absorption Portion

The absorption portion 103 is disposed in the periphery of the introduction portion 102 for the purpose of facilitating the sample introduced into the introduction portion 102 being distributed to the first transportation portions 104 in equal amounts. However, the absorption portion 103 is not necessarily disposed as long as the introduction portion 102 itself is configured to facilitate distribution to the first transportation portions 104 in equal amounts.

First transportation portion and second transportation portion

The first transportation portion 104 is a section configured to transport the sample from the introduction portion 102 or the absorption portion 103 to the mixing portion 105, and the second transportation portion 107 is a section configured to transport the mixture from the mixing portion 105 or the development portion 106 to the bonding portion 108. The materials and the configurations of the first transportation portion 104 and the second transportation portion 107 may be the same or different from each other. In the following description, the first transportation portion 104 and the second transportation portion 107 may be referred to as simply a transportation portion.

Regarding the transportation portion, a porous nitrocellulose film, a porous cellulose film, a nylon film, glass fiber, nonwoven fabric, cloth, and the like can be used, and so-called filter paper is preferable. In the same manner as the above-described introduction portion 102, the surface of the transportation portion can be appropriately selected after distinguishing whether the sample is hydrophilic or hydrophobic. Further, in the case in which the transportation portion has a structure contained in the detection target and a hydrogen-bondable site, it is particularly preferable that treatment to suppress non-specific adsorption be performed. Examples of the treatment to suppress non-specific adsorption include the case in which a surfactant is disposed and the case in which inactive protein is disposed, and the treatment method may be selected in accordance with the characteristics of the detection target in the sample.

In the present embodiment, a plurality of transportation portions are included. Therefore, strips corresponding to the respective transportation portions are equidistantly arranged under the introduction portion, and a hydrophobic resin, for example, a polyethylene or a fluororesin is disposed between the respective strips so as to serve as a partition. Alternatively, a strip is linearly coated with a solution composed of the above-described resin. Consequently, interference of liquids serving as the sample between equidistantly adjacent strips can be prevented.

Mixing Portion

The plurality of mixing portions 105 include the respective signaling substances that differ from each other in the amount. Preferably, the mixing portion 105 has a configuration in which the influent sample and the signaling substance are homogeneously dispersed.

Signaling Substance

Regarding the signaling substance, a material that is selectively bonded to the bonding substance described later and that can provide a signal can be used. In the case in which the signaling substance cannot provide a signal, regarding the signaling substance in the present embodiment, the above-described detection target or an analogue thereof containing a labeling material that can provide a signal in response to a stimulus from the outside may be used. Specifically, the signaling substance is preferably a compound in which a site to be hydrogen-bonded to the bonding substance is not impaired or in which at least a site to be bonded to the bonding substance is left remaining. Further, it is preferable that the signaling substance have the same degree of solubility or dispersibility, as the detection target, into the solvent in which the detection target is dissolved or dispersed, and it is preferable that a material having no association property with the detection target be selected.

Labeling Material

The labeling material in the present embodiment has to make it possible to electrically or optically measure or observe the presence of the signaling substance. The purpose can be achieved not only by the labeling material itself having the characteristics that can be electrically or optically measured or observed but also by adding a reaction group that can be labeled when a labeling step is added as a latter stage, as the situation demands.

When the signaling substance is optically detected, a light-emitting substance, for example, a metal colloid, a dye, a pigment, or a fluorescent substance, can be used as the labeling material. When optical measurement is performed, a similar metal colloid, a carbon-based material, or the like, which have changeable electrical conductivity, or a material that can be provided with a magnetic property may be adopted.

Development Portion

The development portion 106 is disposed for the purpose of mixing the detection target and the signaling substance in the mixture so as to be homogeneously dispersed while the mixture influent from the mixing portion 105 moves to the second transportation portion 107.

It is preferable that the development portion 106 have a larger mesh than the filter material used in the first transportation portion 104. Further, it is preferable that the mesh sizes of the filter materials increase in the order of first transportation portion 104<mixing portion 105<development portion 106. The mesh size may be selected with reference to the filtering time shown in JIS P 3810. For example, in the case in which the freeness of the first transportation portion 104 is 250 seconds, a material of 160 seconds may be selected for the mixing portion 105, and a material of 100 seconds or less may be selected for the development portion 106. This is because, for the purpose of homogeneously mixing the detection target and the signaling substance in the sample in the development portion 106, the flow rate of the sample is temporarily reduced so as to optimize the degree of mixing.

Meanwhile, for the purpose of smoothly transporting the homogeneously mixed mixture to the bonding portion 108, on the contrary, it is preferable that a material having a larger filtering time than the development portion 106 be selected as the filter material used for the second transportation portion 107.

Bonding Portion

The material for forming the bonding portion 108 has to firmly adhere to the bonding substance such that the bonding substance does not flow out of the bonding portion to other sections and, in addition, the bonding substance is preferably selected from materials having no adsorbing property relative to a capture site that captures the detection target or the signaling substance. For example, in the case in which the bonding substance is a resin that can perform molecular recognition, an adhesive that adheres to but does not dissolve into the resin has to be used, and a casein-based adhesive is suitable for use.

Bonding Substance

The bonding substance according to the present embodiment is a substance that can selectively bonded to at least the detection target and the signaling substance. In the present specification, “bonding” is a concept including not only chemical bonds, for example, a hydrogen bond, a covalent bond, and an ionic bond but also physical adsorption with a lower bonding force than the chemical bond. Specific examples include synthetic polymers, antigens, antibodies, and proteins.

Preferably, the bonding substance according to the present embodiment has, in the molecular structure, a site that can be hydrogen-bonded to the detection target or the signaling substance serving as a low-molecular-weight substance, and the atomic arrangement and the spatial configuration of the site in the bonding substance is in accord with those of the site in the detection target or the signaling substance with respect to at least two places so as to mutually complement the hydrogen bond. In addition, if the bonding substance has a shape that spatially surrounding the detection target or the signaling substance, the ability to capture the detection target or the signaling substance can be further improved.

Examples of the site that can form a hydrogen bond include fluorine, oxygen, and nitrogen, which have a high degree of electronegativity, and hydrogen or the like covalent-bonded to such an atom. In this regard, “both the detection target or signaling substance and the bonding substance mutually complement a hydrogen bond” refers to the state in which, for example, when any one of the site of the detection target and the site of the bonding substance includes an atom having a high degree of electronegativity, the other includes hydrogen covalent-bonded to such an atom.

In the above description, the bonding substance has been explained centering on the hydrogen bond, but the same applies when the hydrogen bond is partly or entirely substituted with a covalent bond or an ionic bond.

Checking Portion

In the present embodiment, the checking portion 109 is configured such that it is possible to check whether the sample introduced into the introduction portion reaches the bonding portion.

The checking portion 109 is a section in which the sample introduced into the introduction portion 102 is physically adsorbed due to chromatographic migration and, in addition, an unreacted signaling substance that is not adsorbed by the bonding portion 108 is absorbed and removed. Regarding the material for forming the checking portion 109, water-absorbing materials, for example, cellulose filter paper, nonwoven fabric, cloth, and cellulose acetate, are used.

In this regard, semi-quantification of the detection target may be performed in the checking portion as long as flow paths of the sample are configured to be separated from each other until the checking portion is reached.

Additive

In addition to the signaling substance, surfactants, pH adjusters, organic solvents, buffer salts, and the like may be applied by coating or added in advance in combination. A material that adjusts bonding of the detection target and the signaling substance may be applied by coating in combination with the signaling substance.

Detection Method

The detection method according to the present embodiment includes at least steps of (1) to (4) described below.

(1) A step of distributing the sample containing the detection target in equal amounts

(2) A step of providing a mixture by mixing each of the samples distributed in equal amounts and the signaling substance

(3) A step of bringing the resulting mixture and the bonding substance that can selectively bonded to the detection target and the signaling substance into contact with each other

(4) A step of measuring a signal provided by the signaling substance bonded to the bonding substance

The detection target can be simply detected by using the detection method according to the present embodiment.

### EXAMPLES

### Example 1

In the present example, an urethane open-cell sponge in a square shape having a size of 18 mm×10 mm with a thickness of 3 mm was used for the introduction portion of the sample, and on the bottom surface, three filter paper strips (first transportation portions) 104 composed of nitrocellulose with a width of 5 mm were arranged in parallel. The filter paper used for the strip was selected on the basis of the freeness measurable in conformity with JIS P 3810. In the present example, filter paper with a filtering time of 200 seconds was used. The fiber surface of the filter paper was subjected to water repellent finish by using a surfactant for the purpose of preventing adhesion of the detection target due to penetration of a test liquid. In the present example, three first transportation portions were disposed, and spaces between the transportation portions from the portion under the sponge of the introduction portion to the checking portion were filled with polyethylene wax. A pad (mixing portion) 105 in which a membrane with a filtering time of 180 seconds was impregnated with a signaling substance that was prepared by adding a labeling agent to a detection target in the sample was disposed so as to be overlapped on the strip of the first transportation portion 104. Further, a membrane (development portion) 106 with a filtering time of 100 seconds was overlapped thereon. Subsequently, a membrane 107 with a filtering time of 200 seconds that was the same as the filtering time of the first transportation portion 104 was further laid thereunder. These membranes of the first transportation portion 104, the mixing portion 105, the development portion 106, and the second transportation portion 107 were overlapped without adhering to each other. Thereafter, fixing was performed by using tape or the like from above so as to maintain close contact.

The filtering time of each site was set such that first transportation portion 104>mixing portion 105>development portion 106 applied and, in addition, second transportation portion 107>development portion 106 applied. In particular, the filtering time of the second transportation portion 107 was larger than the filtering time of the development portion 106. Therefore, homogeneous mixing of the signaling substance and the detection target was facilitated, and competitive adsorption of the detection target and the signaling substance in the bonding portion at the latter stage could be accurately performed.

The sample containing mixed detection target and signaling substance was transported by the strip (second transportation portion) 107 due to capillarity and reached the bonding portion 108. The bonding portion 108 carried resin particles or a thin film containing hydrogen-bonding sites that were able to specifically adsorb to the detection target and the signaling substance, and the hydrogen-bonding sites were bonded to the detection target and the signaling substance in accordance with the mixing ratio of these. When a water-soluble dye described later is used as the labeling material of the signaling substance, in the case in which the amount of the detection target is small, adhesion of the signaling substance increases, and the bonding portion is colored. In an opposite case, coloring is not observed or visibility is poor.

In this regard, in the case in which the detection target is discharged from a living body, the degree of change in the range of concentration in accordance with the state of the living body is known. Therefore, it is better that the amounts or the concentrations of the signaling substance disposed in a plurality of mixing portions 105 be changed exponentially such as 2 times and 4 times with reference to the normal concentration, and preferably it is increased by the exponent of 10 such as 10 times and 100 times.

Meanwhile, in the case in which the sample is human urine and 8OHdG (8-oxo-2′-deoxyguanosine) shown in FIG. 4 that is an oxidation metabolite of DNA is detected as a low-molecular-weight compound contained in the urine, a water-soluble dye is bonded to 8OHdG and used as the signaling substance. The pad (mixing portion) 105 is impregnated with the resulting signaling substance and dried so as to be used. In the present embodiment, gold nano-colloidal particles with a diameter of 40 nm were used as a coloring material and bonded to 8OHdG described above by a common method so as to be used. Next, 0.18 ml each of signaling substance solutions in which the molar concentrations of the signaling substance were adjusted to 3 μM, 300 nM, and 30 nM in accordance with the three straps prepared as described above were prepared. The pads (mixing portions) 105 in lines A, B, and C were prepared by being impregnated with the respective solutions and dried.

A polymer produced by copolymerizing a derivative of phenoxazine shown in FIG. 5 and an acrylic acid monomer was used as the bonding substance disposed in the bonding portion. The resulting polymer was fixed to a nitrocellulose-based membrane with a filtering time of 200 seconds by using casein so as to form the bonding portion 108. The number of molecules that can perform the above-described adsorption in the bonding portion is determined in accordance with the covering power of the labeling material contained in the signaling substance used. In the present example, gold colloidal particles with a diameter of 40 nm were used and, therefore, 10⁷ (/μm²) of bonding substance per unit area of the bonding portion 108 had to be disposed. In the present example, polymer adjusted to 2×10⁹ (/μm²) per unit area was used. This polymer was set in the form of impregnation of a midpoint of the strip of the second transportation portion 107. Further, a polymer provided with the bonding substance in an amount larger than that in the bonding portion 108 was prepared downstream of the bonding portion 108 so as to serve as the checking portion 9.

A sample was formed such that the concentration of 8OHdG in artificial urine having a pH adjusted to 6.8 became 10 nM, 200 nM, or 1,500 nM, and a total amount of 0.6 ml of each sample was placed into the introduction portion of a semi-quantitative measuring system (detection kit) of a low-molecular-weight compound described above, and the detection kit was maintained in a horizontal position. After a lapse of several minutes while the horizontal position was maintained, in the case in which the sample of 10 nM was introduced, the bonding portion of only the line of C was not colored, and bonding portions of only the lines of A and B were colored blue and, therefore, it was found that the concentration of the sample was 10 nM. In the case of the sample of 200 nM, only the line of A was colored, and in the case of 1,500 nM, the line B was slightly colored. When the sample of 1,000 nM was introduced, only the line of A was dyed deeply compared with the case of 1,500 nM. Therefore, it could be ascertained that when the order of the concentration was the same, dyeing became deeper as the amount of the detection target decreased.

### Example 2

A system was formed in the same manner as example 1 except that a partition plate 110 was disposed inside the sponge layer of the introduction portion 102 so as to equalize the permeation areas. In addition, a member (absorption portion) 103 that could absorb any sample overflowing the sponge was disposed in the periphery of the introduction portion 102. This member was composed of a known high-molecular-weight absorber or the like, and when a large amount of sample was introduced into the introduction portion 102 or when the sample remained on the edge of the introduction portion, the sample was absorbed quickly. Consequently, quantitativeness of the introduction portion 102 could be ensured. In the case in which three lines were disposed for detecting the detection target, two partition plates 110 were disposed inside the introduction portion 102, and the space partitioned into three parts were filled with the same material as the sponge in the upper portion.

Further, the signaling substance and the capture polymer used in example 1 were disposed in the same manner. Next, three types of samples were prepared and measurement was performed in the same manner as in example 1.

In this case, regarding the detection accuracy, the same result was obtained, and it was further ascertained that even when the detection kit was inclined from the horizontal position, the results did not change, and even when slight differences occurred in operation of the kit, distribution in equal amounts was performed.

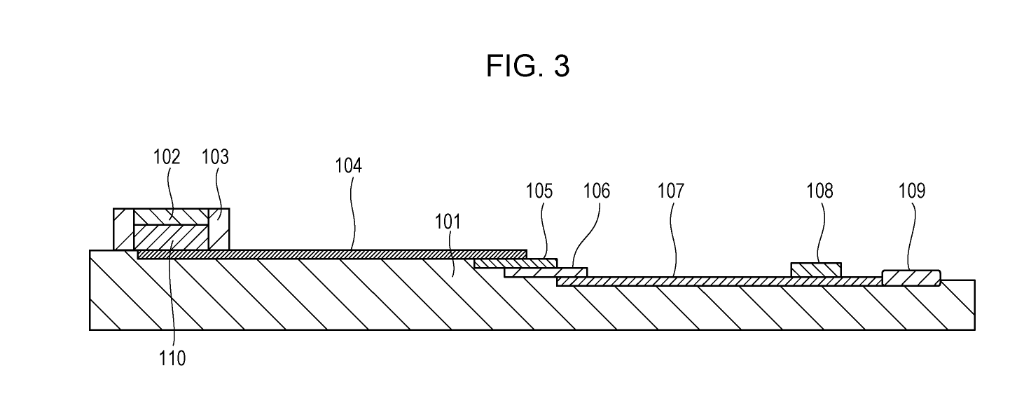

In FIG. 3, the partition plate 110 is lower than the height of the sponge layer of the introduction portion 102. However, there is no problem when the height of the partition plate 110 is equal to the height of the sponge. Alternatively, rectangular frames configured to have the same amount may be prepared rather than the partition plate and disposed on the first transportation portion 104.

According to the detection kit of the present invention, substances to be fixed are reduced than before, degree of freedom increases in selection of the material to be used. Therefore, the configuration is simpler, and the degree of freedom in kit design for a measurement target is higher.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A detection target detection kit comprising,
an introduction portion into which a sample containing the detection target is introduced;
a plurality of mixing portions each configured to provide a mixture of the sample that is introduced into the introduction portion and that is distributed and a signaling substance that can provide a signal; and
bonding portions each including a bonding substance that can be selectively bonded to the detection target and the signaling substance in each of the mixtures provided in the plurality of mixing portions,
wherein the amounts or concentrations of the signaling substance disposed in the plurality of mixing portions are different from each other.

2. The detection kit according to claim 1, wherein the signaling substance is a substance in which a labeling material that can provide a signal is added to the detection target.

3. The detection kit according to claim 1, wherein the coupling constant of the detection target with respect to the bonding substance is equal to the coupling constant of the signaling substance.

4. The detection kit according to claim 1, wherein an absorption portion that absorbs the sample introduced into the introduction portion is disposed in the periphery of the introduction portion.

5. The detection kit according to claim 1, wherein a plurality of first transportation portions configured to transport the sample are disposed between the introduction portion and the mixing portions.

6. The detection kit according to claim 4, wherein the amounts of the sample distributed to the plurality of first transportation portions from the introduction portion are equal to each other.

7. The detection kit according to claim 1, wherein a plurality of second transportation portions configured to transport the mixtures are disposed between the mixing portions and the bonding portions.

8. The detection kit according to claim 1, wherein development portions configured to homogeneously disperse the detection target and the signaling substance in the mixtures provided in the mixing portions are disposed between the mixing portions and the second transportation portions.

9. The detection kit according to claim 1 comprising a checking portion capable of determining whether the sample introduced into the introduction portion reaches the bonding portions by detecting the sample containing the detection target and the signaling substance that are not bonded in the bonding portions.

10. The detection kit according to claim 1, wherein each of the detection target and the signaling substance has a hydrogen-bondable site and the bonding substance has a site that can be hydrogen-bonded to the hydrogen-bondable site.

11. The detection kit according to claim 1, wherein the bonding substance is a synthetic polymer.

12. The detection kit according to claim 1, wherein the detection target is at least one of steroid-based hormones, amine-based hormones, nucleosides, and analogues thereof.

13. The detection kit according to claim 1, wherein the detection target is 8-oxo-2′-deoxyguanosine.

14. The detection kit according to claim 1, wherein any one of the detection target and the bonding substance is an antigen and the other is an antibody.

15. The detection kit according to claim 1, wherein the amounts or concentrations of signaling substance disposed in the plurality of mixing portions differ from each other by an exponent of 10.

16. A detection method for detecting a detection target comprising the steps of:
distributing a sample containing the detection target;
providing a plurality of mixtures by mixing the distributed sample and a signaling substance that can provide a signal;
bringing the plurality of mixtures into contact with a bonding substance that can be selectively bonded to the detection target and the signaling substance; and
measuring a signal provided by the signaling substance bonded to the bonding substance.
